(19) United States
(12) Patent Application Publication
(10) Pub. No.: US 2022/0176054 A1
(43) Pub. Date: Jun. 9, 2022

(54) Intubation Shield

(71) Applicant: Inline Plastics Corp., Shelton, CT (US)

(72) Inventors: Victor Ivenitsky, South Salem, NY (US); Raghav Kharbanda, New Haven, CT (US); Kevin Mello, Monroe, CT (US); Jerzy LaSota, Newington, CT (US); Gregory Jimenez, Ansonia, CT (US); Susanna Mellen, Stratford, CT (US)

(73) Assignee: Inline Plastics Corp., Shelton, CT (US)

(21) Appl. No.: 17/541,535

(22) Filed: Dec. 3, 2021

Related U.S. Application Data
(60) Provisional application No. 63/121,095, filed on Dec. 3, 2020.

(51) Int. Cl. A61M 16/04

(52) U.S. Cl. CPC A61M 16/0488; A61M 2205/02

(57) ABSTRACT

An intubation shield having a viewing panel and a front opening surrounded by a first side wall, a second side wall, a back wall and a front dome wall configured to cover at least a portion of the head, the neck and/or the upper torso of a patient to form a barrier between the covered parts of the patient and the upper body of the practitioner, wherein the viewing panel allows for uninterrupted visibility of an anatomical feature of the patient, and the front opening allows the body of the patient to be inserted while minimizing the droplet contamination, and a first arm hole on the first side wall and a second arm hole on the second side wall to increase the freedom of movement of the practitioner while performing medical procedures.

### CROSS-REFERENCE TO RELATED APPLICATION

The present application claims the benefit of priority to U.S. Provisional Patent Application Ser. No. 63/121,095 filed on Dec. 3, 2020, which is herein incorporated by reference in its entirety.

### FIELD OF THE DISCLOSURE

The present disclosure generally relates to a medical apparatus and methods for using the same and, more particularly to, intubation shields and their use and, still further, to intubation shields which are made using thermoforming techniques from materials such as polyethylene terephthalate (PET).

### BACKGROUND OF THE DISCLOSURE

Various intubation boxes have been developed for medical use. Use of such intubation boxes has increased during the coronavirus pandemic. However, certain of these prior intubation boxes are rigid, heavy and difficult to move, which limits both, the placement over the patient and the movement of the practitioner's arms. Specifically, the limited arm movement makes the present intubation boxes difficult to use or physically awkward during emergencies where rapid intubation is required. Moreover, these current intubation boxes are often made from Plexiglas and need to be sterilized and require a 20-30 minute rest time before they are ready for use. Still further, the seams of these current intubation boxes limit visibility of the patient. Yet another disadvantage of these current intubation boxes is that they are not stackable or nestable which increases the space required for shipment and storage and therefore, can limit the number of units that are available in close proximity to emergency rooms.

Additionally, certain of the current intubation boxes are constructed out of a thin plastic membrane placed on a rigid supporting structure or frame. These intubation boxes inhibit the visibility of the patient if the plastic membrane folds over itself or crumbles/creases, are difficult to move, can be easily ruptured increasing the risk of droplet contamination and restrict the freedom of movement of the arms of the patient due to the thin plastic membrane being inflexible if the plastic membrane is pulled tight over the supporting structure to improve visibility. Also, the supporting structures used in these boxes can be difficult to assemble and obscures patient visibility.

Based on the foregoing, a need exists for intubation shields, that are flexible, light, and easy to move, while increasing the freedom of movement of the practitioner's arms. Furthermore, there exists a need for intubation shields that avoid seams or structure that limit the patient visibility. There is also a need for intubation shields that are stackable to increase the availability of intubation shields in close proximity to emergency rooms and patient rooms where they could be used. The is also a need for intubation shields which can be cost-effectively manufactured and thus can be disposed after use instead of sterilized, thereby decreasing the time and effort spent on maintenance and preventing infections that can be caused by improper sterilization. Thus, an interest exists for an improved intubation shield for medical procedures. The aforementioned inefficiencies and opportunities for improvement are addressed and/or overcome by the assemblies, systems, and methods of the present disclosure.

### SUMMARY OF THE DISCLOSURE

The present invention cures the deficiencies noted above by providing an intubation that is flexible, stackable, lightweight, and easy to move. Moreover, the presently disclosed intubation shields can include a viewing panel to improve the visibility of the practitioner and arm holes arranged and configured to increase the freedom of movement of the arms of the medical practitioner.

The present disclosure also provides intubation shields which can be made using thermoforming techniques from materials such as polyethylene terephthalate (PET). More specifically, disclosed are thermoformed intubation shields which are adapted and configured to be placed over the head and portions of the neck, torso or both of a patient quickly. The disclosed shields include a flat viewing panel to improve the visibility of the oropharyngeal airway and with arm holes that are positioned on the sides of the intubation shield to maximize the freedom of movement of the arms of the practitioner performing an intubation or other procedures on the head, neck or upper torso of the patient.

The exemplary embodiments disclosed herein are illustrative of an intubation box. In accordance with some embodiments, the intubation shield can include a front opening and a viewing panel, surrounded by a first side wall, a second side wall, a back wall and a front dome wall. The intubation shield can be configured to cover the head, portions of the neck and portions of the upper torso of a patient to form a barrier between the covered parts of the patient and the upper body of the practitioner.

In accordance with certain embodiments, the intubation shield can include arm holes that are positioned on the first and the second side walls that allow the practitioner to maximize the freedom of movement of the practitioner performing an intubation or other procedures on the head, neck or upper torso of the patient. Preferably, the arm holes can be an oblong or slotted hole to allow for greater movement.

Preferably, the intubation shield can move with the practitioner to provide a barrier between the upper body of the patient and the practitioner. For example, the intubation shield is lightweight and can move when the practitioner moves his arms and can momentarily lift above the bed level while maintaining the barrier between the upper body of the patient and the practitioner. The gaps at the bed level may not be a concern when the shield shifts up in response to the movement of the arms of the practitioner. In accordance with some embodiments, the intubation shield can be thermoformed to reduce the weight.

In certain embodiments, the intubation shield can be disposable and recyclable. For example, the intubation shield can be a thermoformed PET (polyethylene terephthalate) intubation box, which can be disposed of and recycled. In accordance with some embodiments, due to its ease of use and lightweight construction, the thermoformed PET intubation shield can travel with the patient in contrast to traditional intubation boxes made using Plexiglas. In accordance with some embodiments the intubation shield can be reused for the same patient or disinfected and used for different patient.

In accordance with some embodiments, the intubation shield can be a soft cornered shield with the viewing panel to eliminate visibility issues caused by seams and horizontal walls. In accordance with some embodiments, the intubation shield can include a slight flare on the walls to allow for nesting of the intubation shields while the intubation shields are shipped and/or stored.

Any combination or permutation of features, functions and/or embodiments as disclosed herein is envisioned. Additional advantageous features, functions, and applications of the disclosed systems, methods and assemblies of the present disclosure will be apparent from the description which follows, particularly when read in conjunction with the appended figures. All references listed in this disclosure are hereby incorporated by reference in their entireties.

### BRIEF DESCRIPTION OF DRAWINGS

Features and aspects of embodiments are described below with reference to the accompanying drawings, in which elements are not necessarily depicted to scale.

Exemplary embodiments of the present disclosure are further described with reference to the appended figures. It is to be noted that the various features, steps, and combinations of features/steps described below and illustrated in the figures can be arranged and organized differently to result in embodiments which are still within the scope of the present disclosure.

To assist those of ordinary skill in the art in making and using the disclosed assemblies, systems and methods, reference is made to the appended figures, wherein:

FIGS. 1, 2 and 3 depict a top view, a front view and a side view, respectively, of an intubation shield constructed in accordance with an embodiment of the present invention;

FIGS. 4, 5, 6, 7, 8 and 9 depict a front view, a side view, a back view, a top view, a first perspective view and a second perspective view, respectively, of an intubation shield constructed in accordance with a second embodiment of the present invention, showing a slotted arm hole; and

FIGS. 10, 11, 12, 13, 14 and 15 depict a front view, a side view, a back view, a top view, a first perspective view and a second perspective view, respectively, of an intubation shield constructed in accordance with a third embodiment of the present invention, showing a first slotted arm hole and a second slotted arm hole and a flare on the wall.

### DETAILED DESCRIPTION OF DISCLOSURE

In the following description, it is understood that terms such as “top,” “bottom,” “outward,” “inward,” “internal,” “external,” and the like are words of convenience and are not to be construed as limiting terms. Reference will be made in detail to exemplary embodiments of the disclosure, which are illustrated in the accompanying figures and examples. Referring to the drawings in general, it will be understood that the illustrations are for the purpose of describing particular embodiments of the disclosure and are not intended to limit the same.

Referring now to the drawings, wherein like parts are marked throughout the specification and drawings with the same or similar reference numerals. Drawing figures are not necessarily to scale and in certain views, parts may have been exaggerated for purposes of clarity.

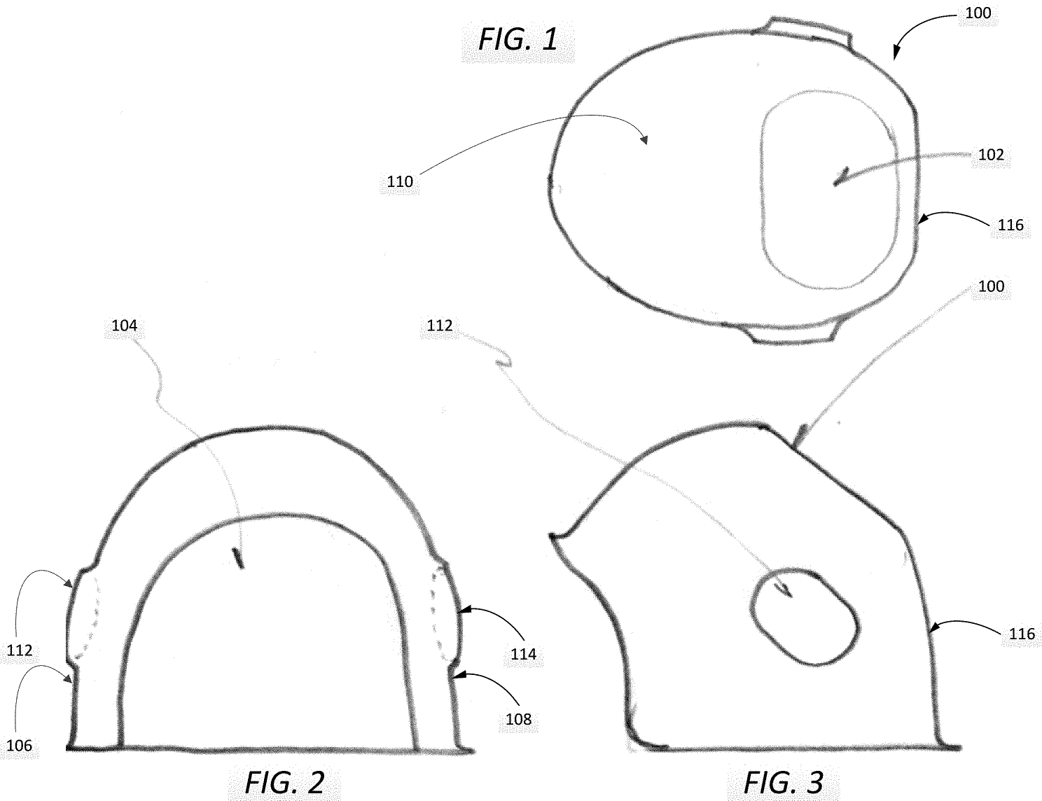

FIGS. 1, 2 and 3 depict an intubation shield 100 constructed in accordance with an embodiment of the present invention. In an embodiment, the intubation shield 100 can include a viewing panel 102 configured to allow an uninterrupted view of an anatomy of a patient, a front opening 104 configured to minimize the droplet contamination while allowing the intubation shield 100 to cover portions of the head, the neck and/or the torso of the patient providing a barrier between the upper body of the patient and a practitioner during a medical procedure. The intubation shield 100 can be formed by a first side wall 106, a second side wall 108, a back wall 116 and a dome wall 110. The first side wall 106 can include a first arm hole 112 and the second side wall 108 can include a second arm hole 114 configured to allow the practitioner maximum freedom of movement while performing a medical procedure, such as an intubation, on a patient. For example, positioning the first arm hole 112 and the second arm hole 114 on the first side wall 106 and the second side wall 108 respectively can allow the practitioner greater flexibility in the elbows compared to positioning the holes on the back wall 116.

In some embodiments, the first arm hole 112, the second arm hole 114 or both can be oblong or slotted or a combination thereof to allow increased freedom of movement for the practitioner while restricting the aerosol or droplet contamination for the practitioner performing the medical procedure. In some embodiments, the intubation shield 100 can have soft corners between the viewing panel 102, the front opening 104, the first side wall 106, the second side wall 108, the back wall 116 and the dome wall 110 to improve the visibility for the practitioner by eliminating seams. For example, the intubation shield 110 can be thermoformed with rounded contours instead of seams at joints between the various parts of the shield to improve visibility for the practitioner while viewing an anatomy of the patient. In some embodiments, the viewing panel 102 can be a flat panel that can blend with soft contours instead of seams with the first side wall 106, the second side wall 108, the back wall 116 and the dome wall 110.

In some embodiments, the intubation shield 100 can be thermoformed from PET. A thermoformed PET intubation shield can allow the intubation shield 100 to weigh less than alternatives made using Plexiglas, allow greater visibility for the practitioner by eliminating seams and horizontal surfaces, allow recycling of the shields and allow disposal of the shield for reducing the risk of infection.

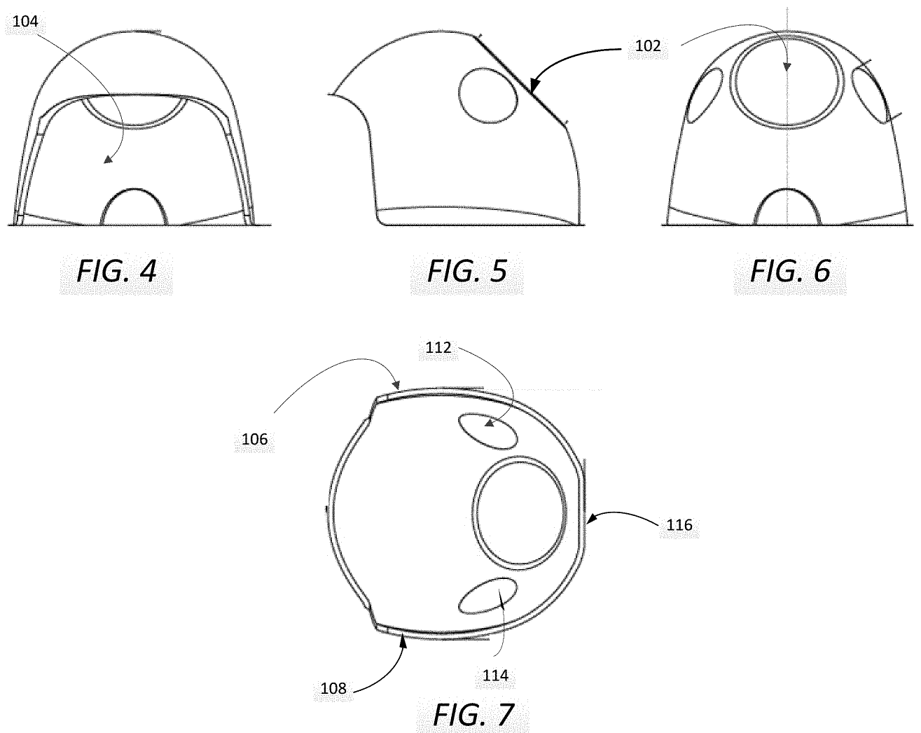

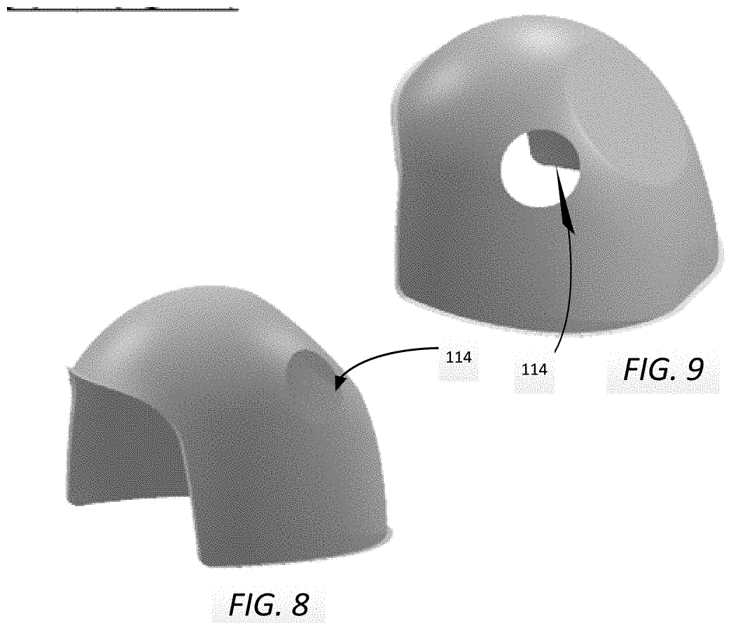

FIGS. 4, 5, 6, 7, 8, and 9 depicts an intubation shield 100 constructed in accordance with a second embodiment of the present invention, showing slotted arm holes. In some embodiments, the back wall 116 can include a flat portion to reduce the distance between the body of the practitioner and the upper body of the patient to increase the freedom of movement of the practitioner while maintaining the barrier between the patient and the practitioner.

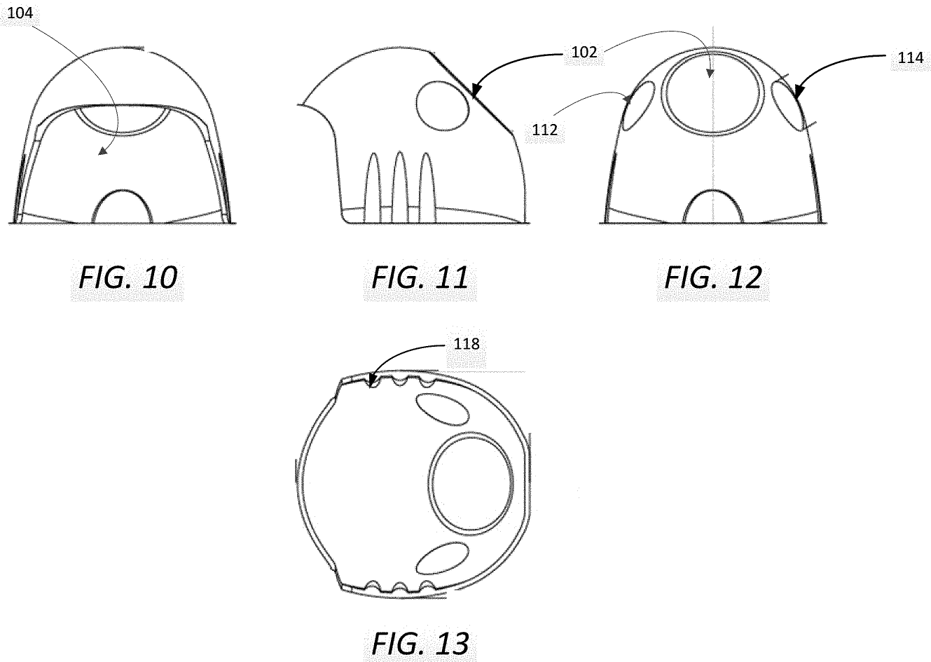

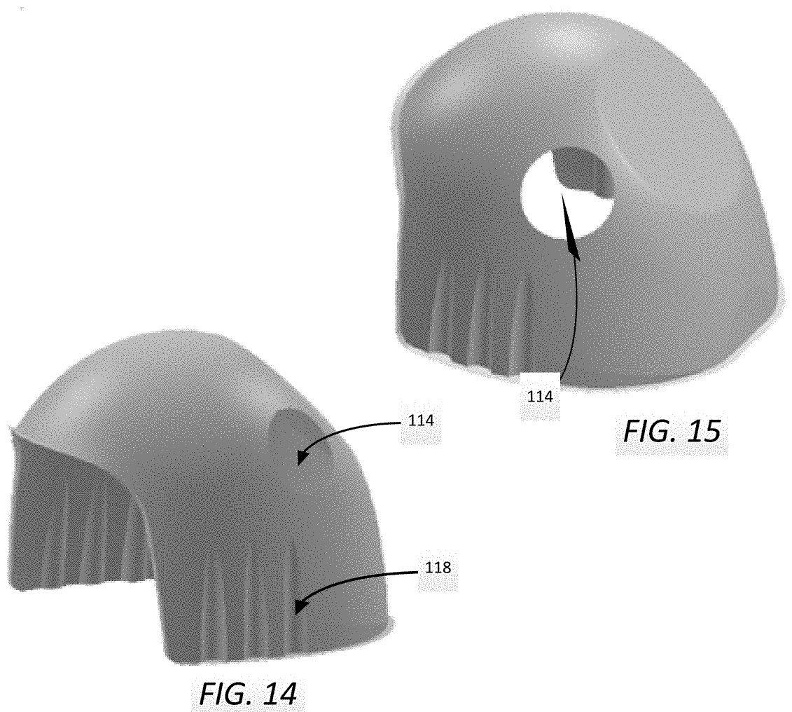

FIGS. 10, 11, 12, 13, 14, and 15 depicts an intubation shield 100 constructed in accordance with a second embodiment of the present invention, showing slotted arm holes and a flare. In some embodiments, the intubation shield 100 can include flares/channels/ribs 118 to allow stacking of the intubation shields 100 during storage and transportation and to improve the rigidity of the shield 100. Those skilled in the art will readily appreciate that the number and arrangement of the flares/channels/ribs 118 can be adjusted in order to provide the desired stiffness of the shield 100 and to ensure the needed visibility. In some embodiments, the back wall 116 can include a flat portion which also allows the intubation shields 100 to be stacked during storage and transportation. In addition, the flares/channels/ribs 118 can allow the practitioner to grip and place the intubation shield 100 or reposition the intubation shield easily.

Although the present disclosure has been described with reference to exemplary implementations, the present disclosure is not limited by or to such exemplary implementations. Rather, various modifications, refinements and/or alternative implementations may be adopted without departing from the spirit or scope of the present disclosure.

1. An intubation shield comprising:
a first side wall, a second side wall, a back wall and a front dome wall configured to cover at least a portion of a patient to form a barrier between the patient and a practitioner, the back wall including a viewing panel that allows for uninterrupted visibility of an anatomical feature of the patient;
wherein a first arm hole is provided in the first side wall and a second arm hole is provided in the
second side wall to increase the freedom of movement of the practitioner while performing medical procedures.

2. The intubation shield of claim 1, wherein the first arm hole and the second arm hole are oblong.

3. The intubation shield of claim 1, wherein the first arm hole and the second arm hole are slotted.

4. The intubation shield of claim 1, wherein the intubation shield is thermoformed.

5. The intubation shield of claim 1, wherein the intubation shield is made using PET (polyethylene terephthalate).

6. The intubation shield of claim 1, wherein the intubation shield is constructed from a light weight material that allows the intubation shield to shift easily when the practitioner is preforming medical procedures to increase the freedom of movement of the patient.

7. The intubation shield of claim 1, wherein the intubation shield has softened contours instead of seams to improve visibility for the physician.

8. The intubation shield of claim 1, wherein the intubation shield has softened contours around the viewing panel instead of seams to improve visibility for the physician.

9. The intubation shield of claim 1, wherein the viewing panel is a flat panel to improve visibility of the anatomy of the patient.

10. The intubation shield of claim 1, wherein the first and second side walls are flared outward with respect to each other in order to allow multiple intubation shields to be stacked.
